(19) United States
(12) Patent Application Publication
(10) Pub. No.: US 2026/0335207 A1
(43) Pub. Date: Oct. 29, 2026

(54) HAIR TREATMENT FORMULATIONS, KITS, AND METHODS FOR PREDETERMINED LEVELS OF HAIR COLOR

(71) Applicant: True Colors Chemistry, Inc., Solana Beach, CA (US)

(72) Inventors: Christopher Birchby, Solana Beach, CA (US); Henry Harper, New Canaan, CT (US); Laura Muollo, Dracut, MA (US); Ju Rhyu, Beverly Hills, CA (US); Abigail Giarrosso, Woburn, MA (US); Omolade Ajayi, Woburn, MA (US); Eric McCue, Encinitas, CA (US)

(21) Appl. No.: 19/657,586

(22) Filed: Apr. 24, 2026

Related U.S. Application Data
(60) Provisional application No. 63/794,826, filed on Apr. 25, 2025.

(51) Int. Cl. A61K 8/41; A61K 8/06; A61K 8/22; A61K 8/44; A61K 8/73; A61K 8/81; A61K 8/92; A61K 8/9789; A61Q 5/10

(52) U.S. Cl. CPC A61K 8/415; A61K 8/062; A61K 8/22; A61K 8/44; A61K 8/73; A61K 8/732; A61K 8/817; A61K 8/922; A61K 8/9789; A61Q 5/10; A61K 2800/88

(57) ABSTRACT

Hair treatment formulations, kits, and methods provide predetermined levels of hair color in safer, cleaner, less complicated alternatives to existing permanent hair dye kits for home or salon use. A hair treatment formulation includes, in some embodiments, a dispersion and an aqueous solution. The dispersion includes a continuous phase and a dispersed phase. The continuous phase includes one or more oils. The dispersed phase includes a pigment precursor; one or more transition metal catalysts; and one or more stabilizers for keeping the dispersed phase dispersed in the continuous phase. The aqueous solution includes an oxidant. The dispersion and the aqueous solution form an oil-in-water emulsion for hair treatment upon combination with each other. The oxidant oxidizes the pigment precursor in accordance with species and/or ratio(s) of the transition metal catalyst(s) in the emulsion to provide a pigment for achieving a predetermined level of hair color with the hair treatment formulation.

### PRIORITY

This application claims the benefit of priority to U.S. Provisional Patent Application No. 63/794,826, filed Apr. 25, 2026, which is incorporated by reference in its entirety into this application.

### BACKGROUND

A permanent hair dye kit for home use typically requires preparation that includes mixing a color cream and an activator to produce the hair dye of the kit. The color cream includes one or more dye precursors such as 1,4-diaminobenzene or 2,5-diaminotoluene and, optionally, one or more related couplers such as 1,3-diaminobenzene, 3-aminophenol, resorcinol, or the like. The activator includes an oxidant such as hydrogen peroxide in a greater concentration than that in household hydrogen peroxide. In addition, either the color cream or the activator can include ammonia, which provides, inter alia, basic reaction conditions during preparation. When the color cream is mixed with the activator, a dye precursor of the color cream is oxidized by the oxidant of the activator to produce a quinone diimine, which then reacts with another dye precursor or a coupler to produce a leuco-dye adduct that ultimately yields the hair dye upon oxidation of the leuco-dye adduct.

While less harmful formulations in permanent hair dye kits such as the foregoing color cream and activator have been in use since the 1970s, the dye precursors, couplers, and resulting hair dyes still pose health risks from preparation through application. Preparation of such hair dyes can be messy and time consuming, thereby adding contact opportunities with the dye precursors and couplers as well as overall exposure time to that already necessitated by the application of the hair dyes. In addition, preparation of such hair dyes is prone to error. What is needed is a safer, cleaner, less complicated alternative to existing permanent hair dye kits for at home or salon use.

Disclosed herein are hair treatment formulations, kits, and methods that provide predetermined levels of hair color in safer, cleaner, less complicated alternatives to existing permanent hair dye kits for home or salon use.

### SUMMARY

Disclosed herein is a hair treatment formulation including, in some embodiments, a dispersion and an aqueous solution. The dispersion includes a continuous phase and a dispersed phase. The continuous phase includes one or more oils. The dispersed phase includes a pigment precursor; one or more transition metal catalysts including at least manganese, iron, zinc, or a combination thereof; and one or more stabilizers for keeping the dispersed phase dispersed in the continuous phase. The aqueous solution includes an oxidant. The dispersion and the aqueous solution form an oil-in-water emulsion for hair treatment upon combination with each other. The oxidant oxidizes the pigment precursor in accordance with species of the transition metal catalyst(s) in the emulsion, ratio(s) of the transition metal catalyst(s) in the emulsion, or both to provide a pigment for achieving a predetermined level of hair color with the hair treatment formulation.

In some embodiments, oxidation of the pigment precursor to the pigment is inhibited in the emulsion by the stabilizer(s). This allows formation of the emulsion, application of the emulsion to hair, and appreciable oxidation of the pigment precursor to the pigment on the hair as opposed to non-inhibited, diffusion-limited oxidation of the pigment precursor to the pigment upon formation of the emulsion.

In some embodiments, oxidation of the pigment precursor to the pigment on the hair bonds the pigment to keratin present in hair shafts of the hair, which contributes to the predetermined level of hair color achieved by the hair treatment formulation.

In some embodiments, the pigment precursor is a catecholamine and the pigment is a polymer having residues selected from various oxidation intermediates of the catecholamine.

In some embodiments, the pigment precursor is levodopoa and the pigment is eumelanin, which has residues selected from those of 5,6-dihydroxyindole (“DHI”) and 5,6-dihydroxyindole-2-carboxylic acid (“DHICA”).

In some embodiments, the pigment has more DHI residues than DHICA residues in accordance with the transition metal catalyst(s) including more manganese.

In some embodiments, the predetermined level of hair color achieved by the hair treatment formulation is ≤2.

In some embodiments, the transition metal catalyst(s) include approximately 4:1 manganese to iron by weight.

In some embodiments, the pigment has more DHICA residues than DHI residues in accordance with the transition metal catalyst(s) including more iron.

In some embodiments, the predetermined level of hair color achieved by the hair treatment formulation is 4-7.

In some embodiments, the transition metal catalyst(s) include approximately 2:1 iron to manganese by weight.

In some embodiments, the oil(s) are selected from coconut oil, fractionated coconut oil, olive oil, argan oil, avocado oil, jojoba oil, sweet almond oil, grapeseed oil, baobab oil, castor oil, macadamia nut oil, marula oil, and sea buckthorn oil.

In some embodiments, the oil(s) include coconut oil or fractionated coconut oil.

In some embodiments, the pigment precursor is present in the dispersed phase as a constituent of Mucuna pruriens seed powder or an extract thereof.

In some embodiments, the stabilizer(s) are selected from dextrin palmitate, sucrose laurate, and dibutyl lauroyl glutamide.

In some embodiments, the stabilizer(s) include dextrin palmitate.

In some embodiments, the oxidant is hydrogen peroxide.

In some embodiments, the aqueous solution further includes one or more viscosity modifiers in a concentration suitable for modifying a viscosity of the aqueous solution.

In some embodiments, the viscosity modifier(s) include konjac gum.

Also disclosed herein is a hair treatment kit including, in some embodiments, a dispensing device including a first compartment and a second compartment, a dispersion in the first compartment, and an aqueous solution in the second compartment. The dispersion includes a continuous phase and a dispersed phase. The continuous phase includes coconut oil or fractionated coconut oil. The dispersed phase includes a constituent of Mucuna pruriens seed powder or an extract thereof as a pigment precursor; manganese, iron, zinc, or a combination thereof as one or more transition metal catalysts; and dextrin palmitate as a stabilizer for keeping the dispersed phase dispersed in the continuous phase. The aqueous solution includes an oxidant and konjac gum in a concentration suitable for modifying a viscosity of the aqueous solution. The dispersion and the aqueous solution form an oil-in-water emulsion for hair treatment upon dispensation from the dispensing device and combination with each other. The oxidant oxidizes the pigment precursor in accordance with species of the transition metal catalyst(s) in the emulsion, ratio(s) of the transition metal catalyst(s) in the emulsion, or both to provide a pigment for achieving a predetermined level of hair color as indicated on packaging of the hair treatment kit.

These and other features of the concepts provided herein will become more apparent to those of skill in the art in view of the accompanying drawings and following description, which describe particular embodiments of such concepts in greater detail.

### BRIEF DESCRIPTION OF THE DRAWINGS

FIG. 1 illustrates oxidation of a pigment precursor of the hair treatment formulation to a pigment in accordance with some embodiments.

FIG. 2A illustrates the pigment as eumelanin with more DHI residues in accordance with some embodiments.

FIG. 2B illustrates the pigment as eumelanin with more DHICA residues in accordance with some embodiments.

FIG. 3 illustrates the pigment as eumelanin aggregated by way of a couple of chelation modes in accordance with some embodiments.

FIG. 4 illustrates a plot of approximate level of hair color vs. ratio of manganese to iron by weight in the hair treatment formulation in accordance with some embodiments.

FIG. 5 illustrates a double-barreled syringe with a mixing tip as a dispensing device for the hair treatment formulation in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Any organic compound specified as its free acid, free base, or salt, as the case might be, is not limited to the specified free acid, free base, or salt. Indeed, any organic compound specified as its free acid can instead be a salt specified by its parent compound or a conjugate base thereof and a counterion of an alkali metal such as sodium or potassium; an alkaline earth metal such as calcium or magnesium; or the like. Any organic compound specified as its free base can instead be a salt specified by its parent compound or a conjugate acid thereof and a counterion of a halide such as chlorine, bromine, or iodine; an oxyanion such as carbonate, nitrate, sulfate, or phosphate; a carboxylate such as formate, acetate, or propionate; hydroxide; or the like. Any organic compound specified as a salt can instead be another salt in accordance with the foregoing; a free acid of the specified salt; or a free base of the specified salt.

Unless defined otherwise, all technical and scientific terms used herein have the same meaning as commonly understood by those of ordinary skill in the art.

As set forth above, a safer, cleaner, less complicated alternative to existing permanent hair dye kits for at home or salon use is needed. Disclosed herein are hair treatment formulations, kits, and methods that provide predetermined levels of hair color in safer, cleaner, less complicated alternatives to existing permanent hair dye kits for home or salon use.

### Hair Treatment Formulations

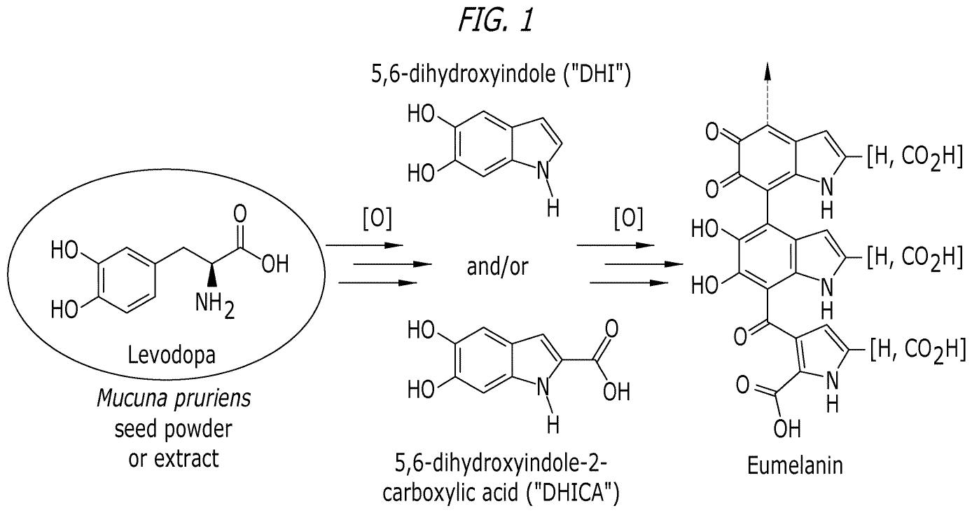

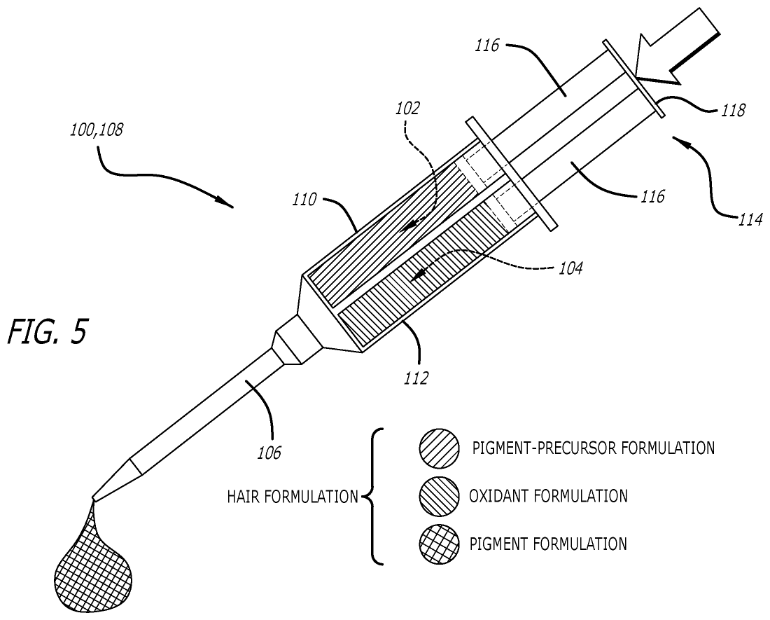

FIGS. 1 and 5 illustrate aspects of the hair treatment formulation in accordance with some embodiments.

The hair treatment formulation includes a pigment-precursor formulation and, in most embodiments, an oxidant formulation, which are configured to form a pigment formulation for hair treatment upon their combination, for example, as shown in FIG. 5. The pigment-precursor formulation includes a pigment precursor and, when present, the oxidant formulation includes an oxidant such that oxidation of the pigment precursor commences upon combination of the pigment-precursor formulation and the oxidant formulation, thereby forming a pigment in the pigment formulation for imparting or restoring color to hair in accordance with the hair treatment formulation. Notably, imparting color to hair can include imparting a lighter or darker color to hair than that associated with restoring color to hair. Indeed, as set forth in more detail below, the hair treatment formulation can provide any of a number of predetermined levels of hair color in accordance with the transition metal catalyst(s) in the pigment formulation thereof.

The pigment-precursor formulation can be an oil-based dispersion and the oxidant formulation can be an aqueous solution as set forth in detail below. However, it should be understood that the inverse is also possible in which the pigment-precursor formulation is an aqueous solution and the oxidant formulation is an oil-based dispersion, albeit with some modifications to that set forth below. For example, when the pigment-precursor formulation is an aqueous solution, the pigment-precursor formulation can be acidic to enhance solubility of the pigment precursor (e.g., levodopa) in the aqueous solution. That, and when the oxidant formulation is an oil-based dispersion, the oxidant can be a solid such as urea-hydrogen peroxide (“UHP”) dispersed throughout the oxidant formulation. Additional base such as the sodium bicarbonate or sodium carbonate set forth below can be added as desired to create alkaline conditions in the pigment formulation upon combination of the pigment-precursor formulation and the oxidant formulation. Whether the pigment-precursor formulation is an oil-based dispersion and the oxidant formulation is an aqueous solution or vice versa, the pigment-precursor formulation and the oxidant formulation are configured to form an emulsion (e.g., an oil-in-water emulsion or a water-in-oil emulsion) as the pigment formulation for hair treatment upon their combination.

The oil-based dispersion includes a continuous phase and a dispersed phase dispersed in the continuous phase. The continuous phase of the oil-based dispersion includes one or more oils. The dispersed phase of the oil-based dispersion includes the pigment precursor and one or more transition metal catalysts for increasing an oxidation rate of the pigment precursor and, as set forth in more detail below, adjusting a character of the pigment for achieving a predetermined level of hair color with the hair treatment formulation. Being that an oxidant such as hydrogen peroxide is not optimally shelf stable above about pH 5, the dispersed phase of the oil-based dispersion preferably includes one or more pH-modifying agents instead of the aqueous solution, if desired, for basifying the aqueous phase of the emulsion formed upon combination of the oil-based dispersion and the aqueous solution. That said, the aqueous solution can include its own pH-modifying agent(s) including, but not limited to, citric acid for acidifying the aqueous solution to about pH 5 or lower for shelf stability prior to the foregoing combination with the oil-based dispersion. Lastly, the dispersed phase of the oil-based dispersion can include one or more stabilizers for keeping the dispersed phase dispersed in the continuous phase.

The oil(s) are selected from at least coconut oil, fractionated coconut oil, which is also known as medium-chain triglyceride (“MCT”) oil, olive oil, argan oil, avocado oil, jojoba oil, sweet almond oil, grapeseed oil, baobab oil, castor oil, macadamia nut oil, marula oil, and sea buckthorn oil.

The pigment precursor can be any catecholamine expected to form one or more 5,6-dihydroxyindoles via oxidation en route to a polymer as the pigment. As shown in FIG. 1, such a catecholamine includes, but is not limited to, levodopa, which forms DHI and DHICA via oxidation en route to eumelanin as the pigment. Levodopa naturally occurs in Mucuna pruriens seeds up to at least about 6% by dry weight, so levodopa can be present in the dispersed phase as a constituent of M. pruriens seed powder, a constituent of an extract of M. pruriens seeds, or a substantially pure form of levodopa derived from M. pruriens seeds. However, it should be understood that levodopa can additionally or alternatively be sourced from any part of M. pruriens as well as any other natural (or genetically modified) plant having tyrosinase including, but not limited to, Stizolobium deeringianum or Vicia faba. Further, levodopa can even be sourced from a synthetic source. Additionally or alternatively, the pigment precursor can include any one or more oxidation intermediates of the catecholamine en route to the pigment such as any one or more stable intermediates of melanogenesis selected from at least leucodopachrome, dopachrome, DHI, and DHICA. Notably, the pigment precursor can additionally or alternatively include any one or more stable intermediates en route to pheomelanin selected from at least 2-S-cysteinyldopa, 5-S-cysteinyldopa, and their corresponding 1,4-benzothiazinyl-alanine metabolites; allomelanin selected from 1,3,6,8-tetrahydroxynaphthalene (“THN”) and 1,8-dihydroxynaphthalene (“DHN”); or a combination thereof.

As set forth below, a combination or admixture of components including the pigment precursor, the transition metal catalyst(s), and the pH-modifying agent(s), if desired, can be mixed into the oil(s), optionally, already including the stabilizer(s). With the understanding that M. pruriens seeds include up to at least about 6% levodopa by dry weight, such an admixture can include M. pruriens seed powder or an extract thereof in a concentration of at least about 20%, 30%, 40%, 50%, 60%, 70%, or 80% by weight inclusive of any intervening concentrations defined by the so-called ones place. Alternatively, the admixture can include M. pruriens seed powder or an extract thereof in a concentration of no more than about 80%, 70%, 60%, 50%, 40%, 30%, or 20% by weight inclusive of any intervening concentrations defined by the so-called ones place. Combining the foregoing open-ended ranges into closed ranges, the admixture can include M. pruriens seed powder or an extract thereof in a concentration of at least about 20% and no more than about 80% by weight, including at least about 30% and no more than about 70% by weight, such as at least about 40% and no more than about 60% by weight, for example, at least about 45% and no more than about 55% by weight. Being that levodopa can be sourced from any natural (or genetically modified) plant having tyrosinase, however, the admixture can alternatively or additionally include a powder or extract of such a plant (or part thereof) in a concentration within one of the foregoing concentration ranges. It should be understood that the concentration of levodopa vehicle (e.g., the M. pruriens seed powder or extract thereof) in the admixture can vary in accordance with the concentration of levodopa in the levodopa vehicle. Indeed, lower concentrations of levodopa in the levodopa vehicle (e.g., <5% levodopa in the M. pruriens seed powder or extract thereof) lead to some of the higher concentrations given for the levodopa vehicle in the admixture, and higher concentrations of levodopa in the levodopa vehicle (e.g., >5% levodopa in the M. pruriens seed powder or extract thereof) lead to some of the lower concentrations given for the levodopa vehicle in the admixture. In step with the latter, substantially pure forms of levodopa lead to lower concentrations of levodopa in the admixture.

The transition metal catalyst(s) respectively include one or more transition metals selected from those in a range of the Periodic Table of the Elements defined by the d-Block and, optionally, the f-Block, which transition metals are often considered inner transition metals. The transition metal(s) can be further selected from those in a range of the Periodic Table of the Elements defined by Groups 3-12 and Periods 4-6, including Groups 4-12 and Periods 4 and 5, such as Groups 6-12 and Periods 4 and 5, for example, Groups 7-12 and Period 4, or some combination of the foregoing Groups and Periods. For example, the transition metal(s) can be selected from titanium, chromium, molybdenum, manganese, iron, cobalt, nickel, copper, silver, and zinc. Notably, such transition metal(s) can assume any of a number of useful oxidation states for increasing the oxidation rate of the pigment precursor. Further, the transition metal(s) as well as the oxidation state(s) thereof (and the counterion(s) associated therewith, as below) can influence the level of hair color and shade. For example, transition metals that directly oxidize the pigment precursor such as iron or copper generate warmer shades of brown with red, orange, or yellow undertones while manganese generates cooler or darker shades of brown or black with more green, blue, or violet undertones. But transition metals such as zinc, which do not directly oxidize the pigment precursor but, notably, stabilize certain reaction intermediates via coordination chemistry, generate warmer shades of brown with red, orange, or yellow undertones.

The transition metal catalyst(s) can be associated with one or more counterions selected from those ranging in character from simple charge-balancing counterions to ligands that form coordination complexes with the transition metal catalyst(s). With respect to ligands, the counterion(s) can be selected from ligands that range from monodentate ligands to multidentate ligands such as bidentate ligands or tridentate ligands that chelate the transition metal catalyst(s) through two or more donor atoms. It should be understood, however, that the character of the counterion(s) in the pigment-precursor formulation or pigment formulation can depend upon various chemical factors. With the foregoing understanding, the counterion(s) can be selected from at least chloride, sulfate, subsulfate, ascorbate, citrate, gluconate, glycinate, and lactate. Continuing with the manganese, iron, and zinc of the foregoing transition-metal example, the counterion can be, for example, gluconate, and the transition metal catalyst(s) can be selected from manganese gluconate, iron gluconate, and zinc gluconate, the transition metal of each of which is in its+2-oxidation state. Notably, the counterion(s) can influence the solubility and, thus, concentration of certain reaction intermediates in the pigment formulation as well as the solvation and, thus, stabilization (e.g., via hydrogen bonding) of those reaction intermediates, thereby influencing reaction rates and levels of hair color and shade.

As set forth below, a combination or admixture of components including the pigment precursor, the transition metal catalyst(s), and the pH-modifying agent(s), if desired, can be mixed into the oil(s), optionally, already including the stabilizer(s). Such an admixture can include the transition metal catalyst(s) in a concentration of at least about 1%, 5%, 10%, 15%, 20%, or 25% by weight inclusive of any intervening concentrations defined by the so-called ones place. Further, the admixture can include the transition metal catalyst(s) in a concentration of at least about 0.1%, 0.2%, 0.3%, 0.4%, 0.5%, 0.6%, 0.7%, 0.8%, or 0.9% by weight inclusive of any intervening concentrations defined by the so-called hundredths place. Alternatively, the admixture can include the transition metal catalyst(s) in a concentration of no more than about 25%, 20%, 15%, 10%, 5%, or 1%, by weight inclusive of any intervening concentrations defined by the so-called ones place. Included in the foregoing, is a concentration of the transition metal catalyst(s) in the admixture of no more than about 0.9%, 0.8%, 0.7%, 0.6%, 0.5%, 0.4%, 0.3%, 0.2%, or 0.1% by weight inclusive of any intervening concentrations defined by the so-called hundredths place. Combining the foregoing open-ended ranges into closed ranges, the admixture can include the transition metal catalyst(s) in a concentration of at least about 0.1% and no more than about 25% by weight, including at least about 0.5% and no more than about 20% by weight, such as at least about 1% and no more than about 15% by weight, for example, at least about 5% and no more than about 10% by weight.

The pH-modifying agent(s) are selected from organic bases and inorganic bases, any of which can be present as part of a buffering system with its conjugate acid(s) when the pH-modifying agent(s) are present. Indeed, it should be understood that, in some embodiments, an exogenous pH-modifying agent is not needed for oxidizing the pigment precursor to the pigment via the transition metal catalyst(s). Notwithstanding the foregoing, an organic base can include, for example, a basic amino acid such as arginine, histidine, or lysine. An inorganic base can include, for example, a hydroxide such as ammonium hydroxide, a carbonate such as sodium bicarbonate, potassium bicarbonate, sodium carbonate, or potassium carbonate, or a phosphate such as sodium dihydrogen phosphate, sodium hydrogen phosphate, or trisodium phosphate. Continuing with the carbonate example, the sodium bicarbonate and sodium carbonate can be part of a carbonate buffering system for modifying the pH of the aqueous phase of the emulsion upon combination of the oil-based dispersion and the aqueous solution as well as maintaining the pH of the aqueous phase of the emulsion so modified. Notably, the pH-modifying agent(s) are in a concentration in the oil-based dispersion suitable for modifying the pH of the aqueous phase of the emulsion upon combination of the oil-based dispersion and the aqueous solution. Further, the pH of the aqueous phase of the emulsion can be greater than about pH 7 upon combination of the oil-based dispersion and the aqueous solution for effectuating oxidation of the pigment precursor. Lastly, in addition to influencing pH, the pH-modifying agent(s) can influence the level of hair color as well as its shade. With respect to organic bases, for example, arginine generates warmer shades of brown with red, orange, or yellow undertones. With respect to inorganic bases, for example, sodium bicarbonate and potassium bicarbonate generate cooler or darker shades of brown with more green, blue, or violet undertones than that generated with sodium carbonate or potassium carbonate.

As set forth below, a combination or admixture of components including the pigment precursor, the transition metal catalyst(s), and the pH-modifying agent(s), if desired, can be mixed into the oil(s), optionally, already including the stabilizer(s). Such an admixture can include the pH-modifying agent(s) in a concentration of at least about 20%, 30%, 40%, 50%, 60%, 70%, or 80% by weight inclusive of any intervening concentrations defined by the so-called ones place. Alternatively, the admixture can include the pH-modifying agent(s) in a concentration of no more than about 80%, 70%, 60%, 50%, 40%, 30%, or 20% by weight inclusive of any intervening concentrations defined by the so-called ones place. Combining the foregoing open-ended ranges into closed ranges, the admixture can include the pH-modifying agent(s) in a concentration of at least about 20% and no more than about 80% by weight, including at least about 30% and no more than about 70% by weight, such as at least about 40% and no more than about 60% by weight, for example, at least about 45% and no more than about 55% by weight.

When present, the stabilizer(s) are selected from at least dextrin palmitate; esterified sugars such as glucose laurate, sucrose laurate, and sucrose stearate; fatty-acid esters such as cetyl palmitate, cetyl ethylhexanoate, cetyl stearate, stearyl ethylhexanoate, stearyl stearate; and dibutyl lauroyl glutamide.

As set forth below, the stabilizer(s) can be mixed into the oil(s), thereby forming the nascent oil-based dispersion of the stabilizer(s) in the oil(s). The nascent oil-based dispersion can include the stabilizer(s) in a concentration of at least about 1%, 5%, 10%, 15%, 20%, or 25% by weight inclusive of any intervening concentrations defined by the so-called ones place. Alternatively, the nascent oil-based dispersion can include the stabilizer(s) in a concentration of no more than about 25%, 20%, 15%, 10%, 5%, or 1% by weight inclusive of any intervening concentrations defined by the so-called ones place. Combining the foregoing open-ended ranges into closed ranges, the nascent oil-based dispersion can include the stabilizer(s) in a concentration of at least about 1% and no more than about 25% by weight, including at least about 1% and no more than about 20% by weight, such as at least about 1% and no more than about 15% by weight, for example, at least about 5% and no more than about 10% by weight.

As set forth below, a combination or admixture of components including the pigment precursor, the transition metal catalyst(s), and the pH-modifying agent(s), if desired, can be mixed into the oil(s) including the stabilizer(s), namely, the nascent oil-based dispersion, thereby forming the oil-based dispersion of the hair treatment formulation. The admixture can have a concentration of at least about 20%, 30%, 40%, 50%, 60%, 70%, or 80% by weight in the oil-based dispersion inclusive of any intervening concentrations defined by the so-called ones place. Alternatively, the admixture can have a concentration of no more than about 80%, 70%, 60%, 50%, 40%, 30%, or 20% by weight in the oil-based dispersion inclusive of any intervening concentrations defined by the so-called ones place. Combining the foregoing open-ended ranges into closed ranges, the admixture can have a concentration in the oil-based dispersion of at least about 20% and no more than about 80% by weight, including at least about 30% and no more than about 70% by weight, such as at least about 40% and no more than about 60% by weight, for example, at least about 45% and no more than about 55% by weight.

The aqueous solution includes water and an oxidant dissolved therein for oxidizing the pigment precursor to the pigment when such an oxidant is desired. Indeed, in some embodiments, an exogenous oxidant is not needed for oxidizing the pigment precursor to the pigment via the transition metal catalyst(s). Notwithstanding the foregoing, the oxidant can be hydrogen peroxide. Additionally or alternatively, the oxidant can include any one or more oxidants selected from organic oxidants and inorganic oxidants other than hydrogen peroxide. An organic oxidant can include, for example, UHP, tert-butyl hydrogen peroxide, or 4-hydroxy-2,2,6,6-tetramethylpiperidin-1-oxyl (“TEMPO”). An inorganic oxidant can include, for example, a percarbonate such as sodium percarbonate; an iodate such as sodium iodate or potassium iodate; a perborate such as sodium perborate; a peroxydisulfate such as ammonium peroxydisulfate, sodium peroxydisulfate, or potassium peroxydisulfate; a bromate such as sodium bromate or potassium bromate; a hypochlorite such as sodium hypochlorite; or a periodate such as sodium periodate or potassium periodate.

As set forth below, the oxidant can be mixed into water before the viscosity modifier(s). When such an aqueous solution includes 3% aqueous hydrogen peroxide as the oxidant, the aqueous solution can include the 3% aqueous hydrogen peroxide in a concentration of at least about 20%, 30%, 40%, 50%, 60%, 70%, or 80% by weight inclusive of any intervening concentrations defined by the so-called ones place. Alternatively, the aqueous solution can include the 3% aqueous hydrogen peroxide in a concentration of no more than about 80%, 70%, 60%, 50%, 40%, 30%, or 20% by weight inclusive of any intervening concentrations defined by the so-called ones place. Combining the foregoing open-ended ranges into closed ranges, the aqueous solution can include the 3% aqueous hydrogen peroxide in a concentration of at least about 20% and no more than about 80% by weight, including at least about 20% and no more than about 70% by weight, such as at least about 20% and no more than about 60% by weight, for example, at least about 30% and no more than about 50% by weight.

The aqueous solution can further include one or more viscosity modifiers in a concentration suitable for modifying a viscosity of the aqueous solution. Indeed, the viscosity of the aqueous solution can be coordinated with that of the oil-based dispersion to facilitate combination of the oil-based dispersion and the aqueous solution in the mixing tip 106 of the dispensing device 100 to form the emulsion. When present, the viscosity modifier(s) are selected from at least one or more gums including konjac gum, sclerotium gum, xanthan gum, carrageenan gum, guar gum, locust bean gum, and chemically modified forms thereof such as hydroxypropyl guar. Additionally or alternatively, the viscosity modifier(s) are selected from at least one or more cellulosics including hydroxypropyl methylcellulose or some other cellulose ether.

As set forth below, the viscosity modifier(s) can be mixed into the aqueous solution including the oxidant, thereby forming the aqueous solution of the hair treatment formulation. The viscosity modifier(s) can have a concentration of at least about 0.5%, 1.0%, 2.0%, 3.0%, 4.0%, or 5.0% by weight in the aqueous solution of the hair treatment formulation inclusive of any intervening concentrations defined by the so-called tenths place. Alternatively, the viscosity modifier(s) can have a concentration of no more than about 5.0%, 4.0%, 3.0%, 2.0%, 1.0 or 0.5% by weight in the aqueous solution of the hair treatment formulation inclusive of any intervening concentrations defined by the so-called tenths place. Combining the foregoing open-ended ranges into closed ranges, the viscosity modifier(s) can have a concentration in the aqueous solution of the hair treatment formulation of at least about 0.5% and no more than about 5% by weight, including at least about 0.5% and no more than about 3% by weight, such as at least about 0.5% and no more than about 2% by weight, for example, at least about 0.5% and no more than about 1% by weight. Notably, depending upon the species of the one or viscosity modifiers, concentrations of the viscosity modifier(s) more than about 1% can form gels instead of solutions.

The oil-based dispersion, the aqueous solution, or both the oil-based dispersion and the aqueous solution can independently include one or more phase-transfer catalysts. Such phase-transfer catalyst(s) facilitate ion shuttling between the oil-based dispersion and the aqueous solution, mixing the oil-based dispersion and the aqueous solution, particularly when a phase-transfer catalyst also functions as a surfactant, or both upon combination of the oil-based dispersion and the aqueous solution. When present, the phase-transfer catalyst(s) are selected from at least quaternary ammonium salts, which, in turn, include at least alkyltrimethylammonium salts such as cetyltrimethylammonium chloride and docosyltrimethylammonium chloride; dialkyldimethylammonium salts such as dicetyldimethylammonium chloride and distearyldimethylammonium chloride; and alkylbenzyldimethylammonium salts, which are also known as benzalkonium salts, such as dodecylbenzalkonium chloride, tetradecylbenzalkonium chloride, hexadecylbenzalkonium chloride, and stearalkonium chloride.

The oil-based dispersion, the aqueous solution, or both the oil-based dispersion and the aqueous solution can independently include one or more emulsifiers to reduce the surface tension between the oil-based dispersion and the aqueous solution allowing them to mix and form the emulsion of the pigment formulation.

The oil-based dispersion, the aqueous solution, or both the oil-based dispersion and the aqueous solution can independently include one or more preservatives. When present, the preservative(s) are selected from at least phenethyl alcohol, sodium benzoate, gluconolactone, potassium sorbate, sodium anisate, sodium phytate, aspen bark extract, willow bark extract, leucidal liquid, propanediol, pentylene glycol, glycerin, ethylhexyl glycerin, caprylyl glycol, and glyceryl caprylate-caprate. Like that set forth above, any preservative of the foregoing preservatives specified as its free acid, free base, or salt, as the case might be, is not limited to the specified free acid, free base, or salt. Any preservative of the foregoing preservatives specified as its free acid can instead be a salt specified by its parent compound or a conjugate base thereof and a counterion of an alkali metal such as sodium or potassium; an alkaline earth metal such as calcium or magnesium; or the like. Any preservative of the foregoing preservatives specified as its free base can instead be a salt specified by its parent compound or a conjugate acid thereof and a counterion of a halide such as chlorine, bromine, or iodine; an oxyanion such as carbonate, nitrate, sulfate, or phosphate; a carboxylate such as formate, acetate, or propionate; hydroxide; or the like. Any preservative of the foregoing preservatives specified as a salt can instead be another salt in accordance with the foregoing; a free acid of the specified salt; or a free base of the specified salt. For example, the preservative(s) can be a combination of phenethyl alcohol, glycerin, and benzoic acid.

The oil-based dispersion, the aqueous solution, or both the oil-based dispersion and the aqueous solution can independently include one or more emollients configured to facilitate spreading the pigment formulation on the scalp or make the pigment formulation feel better on the scalp. When present, the emollient(s) are selected from lipophilic emollients including lauryl lactate, isoamyl laurate, and isopropyl myristate and hydrophilic emollients including those having water-solubilizing polyethylene glycol (“PEG”) moieties such as PEG-7 olivate, PEG-7 glyceryl cocoate, and PEG-8 caprylic/capric glycerides.

The oil-based dispersion, the aqueous solution, or both the oil-based dispersion and the aqueous solution can independently include one or more haircare ingredients that impart haircare benefits. When present, the haircare ingredient(s) are selected from at least tea tree oil, rosemary oil, panthenol, various plant-derived acids including at least lactic acid and salicylic acid, retinol, various peptides, and various antioxidants.

The oil-based dispersion, the aqueous solution, or both the oil-based dispersion and the aqueous solution can independently include one or more hair-penetration ingredients that improve penetration of various ingredients into hair. When present, the hair-penetration ingredient(s) are selected from at least alcohols including diols such as glycols and triols such as glycerin, fatty acids, amino acids, peptides, hydrolyzed proteins, and various surfactants.

Notably, as with the pigment-precursor levodopa that naturally occurs in M. pruriens seeds, components of the hair treatment formulation are preferably sourced from safe, non-toxic natural sources or at least found in such natural sources, thereby ensuring the hair treatment formulation is a safer alternative to existing permanent hair dye kits for at home or salon use. Should one or more components of the hair treatment formulation not be found in such natural sources, the component(s) are preferably previously reviewed and found safe by the Cosmetic Ingredient Review of Washington D.C.

While different levels of hair color can be achieved with the hair treatment formulation through oxidation of different pigment precursors to correspondingly different pigments in the pigment formulation, it has been surprisingly found that such different levels of hair color can be alternatively achieved with a single pigment precursor by varying the transition metal catalyst(s) for the oxidation. Indeed, the character of the pigment resulting from oxidation of a single pigment precursor such as levodopa can be adjusted in accordance with species of the transition metal catalyst(s) in the pigment formulation, ratio(s) of the transition metal catalyst(s) in the pigment formulation, or both. Notably, it has also been found that the character of the pigment resulting from oxidation of the foregoing pigment precursor can also be adjusted in accordance with the ratio(s) of the transition metal catalyst(s) to the pigment precursor in the pigment formulation.

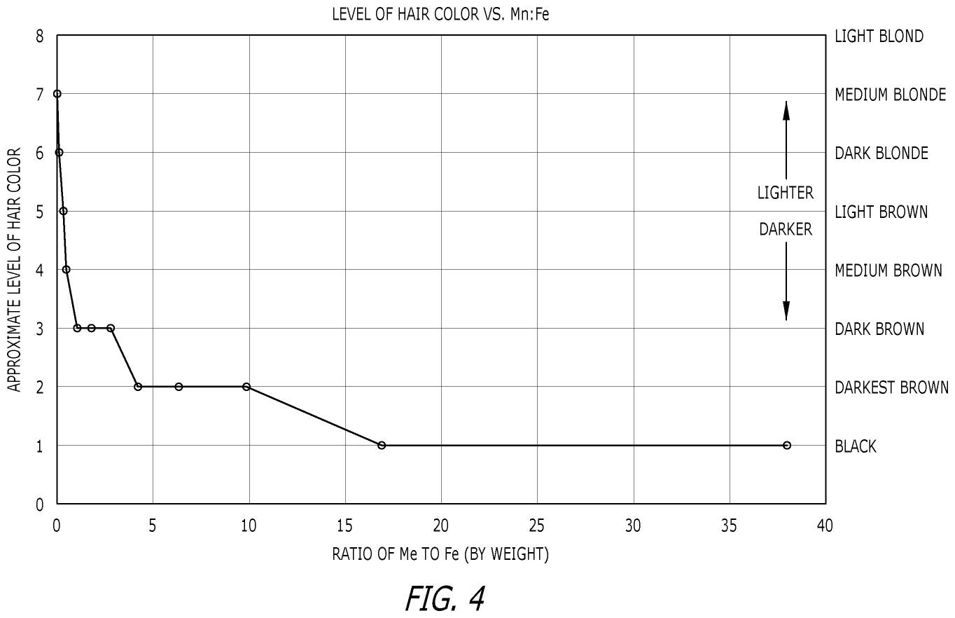

As set forth above, the transition metal catalyst(s) can be selected from at least manganese, iron, and zinc, which can be present as manganese gluconate, iron gluconate, and zinc gluconate, respectively, in the admixture that is added to the oil(s) to form the oil-based dispersion as the pigment-precursor formulation. As further set forth above, the admixture can include such transition metal catalysts in a concentration up to about 5% by weight (or more) whether the admixture includes a single transition metal catalyst or a combination of transition metal catalysts. Taking, in view of the foregoing, manganese gluconate and iron gluconate as examples of the species of the transition metal catalyst(s) that can be adjusted in the pigment formulation resulting from combination of the pigment-precursor formulation and the oxidant formulation, Table 1 provides concentrations for manganese gluconate and iron gluconate in various admixtures, ratios of manganese gluconate and iron gluconate to each other in the foregoing admixtures, and approximate levels of hair color achieved with corresponding hair treatment formulations. As shown in FIG. 4, which illustrates a plot of approximate level of hair color vs. ratio of manganese to iron by weight in the hair treatment formulation, the relationship between the approximate level of hair color achieved with the hair treatment formulation and the ratio of manganese gluconate and iron gluconate to each other in the corresponding admixture resembles a power-law or logarithmic relationship instead of a simple linear relationship.

Table 1. Manganese gluconate and iron gluconate by percent weight in the admixture and corresponding level of hair color achieved with the hair treatment formulation.

| | | | Approx. | Approx. |
|---|---|---|---|---|
| | | Mn:Fe | ratio | level of |
| Wt. % Mn | Wt. % Fe | (by wt.) | Mn:Fe | hair color* |
| 0.000 | 1.220 | 0 | N/A | 7 |
| 0.129 | 1.190 | 0.1082 | 1:10 | 6 |
| 0.258 | 1.159 | 0.2222 | 1:5 | 5 |
| 0.386 | 1.129 | 0.3423 | 1:3 | 5 |
| 0.515 | 1.098 | 0.4690 | 1:2 | 4 |
| 1.030 | 0.976 | 1.0553 | 1:1 | 3 |
| 1.545 | 0.854 | 1.8091 | 2:1 | 3 |
| 2.060 | 0.732 | 2.8142 | 3:1 | 3 |
| 2.575 | 0.610 | 4.2213 | 4:1 | 2 |
| 3.090 | 0.488 | 6.3320 | 6:1 | 2 |
| 3.605 | 0.366 | 9.8497 | 10:1 | 2 |
| 4.120 | 0.244 | 16.8852 | 17:1 | 1 |
| 4.635 | 0.122 | 37.9918 | 38:1 | 1 |
| 5.150 | 0.000 | #DIV/0! | N/A | 1 |

*The hair color level system used across various professional hairdressing brands indicates the following levels of hair color: Level 10: Lightest blonde; Level 9: Very light blonde; Level 8: Light blonde; Level 7: Blonde; Level 6: Dark blonde; Level 5: Light brown; Level 4: Brown; Level 3: Dark brown; Level 2: Darkest brown; and Level 1: Black. (See also, FIG. 4.)

While Table 1 provides ratios of manganese gluconate and iron gluconate to each other in the admixture used to form the pigment-precursor formulation, it should be understood that such ratios can be extended to other pairs of transition metal catalysts in the admixture, which pairs of transition metal catalysts can optionally include one of manganese gluconate or iron gluconate. Further, it should be understood that such ratios can also be extended over a greater range, as desired, to accommodate transition metal catalysts having greater or lesser formula weights, achieve various levels of hair color with the hair treatment formulation, or both. Accordingly, the ratio between a first transition metal catalyst and a second transition metal catalyst in the admixture can be at least about 1:100, 1:90, 1:80, 1:70, 1:60, 1:50, 1:40, 1:30, 1:20, 1:10, or 1:1 inclusive of any intervening ratios defined by the so-called ones place. Alternatively, the ratio between the first transition metal catalyst and the second transition metal catalyst in the admixture can be no more than about 100:1, 90:1, 80:1, 70:1, 60:1, 50:1, 40:1, 30:1, 20:1, 10:1, or 1:1 inclusive of any intervening ratios defined by the so-called ones place. Combining the foregoing open-ended ranges into closed ranges, the ratio between the first transition metal catalyst and the second transition metal catalyst in the admixture can be at least about 1:100 and no more than about 100:1, including at least about 1:50 and no more than about 50:1, such as at least about 1:30 and no more than about 30:1, for example, at least about 1:20 and no more than about 20:1 or at least about 1:10 and no more than about 10:1.

While embracing some theoretical flexibility, adjusting the species of the transition metal catalyst(s) in the pigment formulation, the ratio(s) of the transition metal catalyst(s) in the pigment formulation, or both can affect the oxidation of the pigment precursor and, thus, distribution of various oxidation intermediates of the pigment precursor in the pigment formulation. This, in turn, can affect polymerization of the pigment as the distribution of the foregoing oxidation intermediates controls their availability for incorporation into the pigment, again, a polymer, as residues thereof. Indeed, subsequent to oxidation of the pigment precursor and polymerization of the resulting oxidation intermediates, the resulting polymer has residues selected from the oxidation intermediates of the pigment precursor.

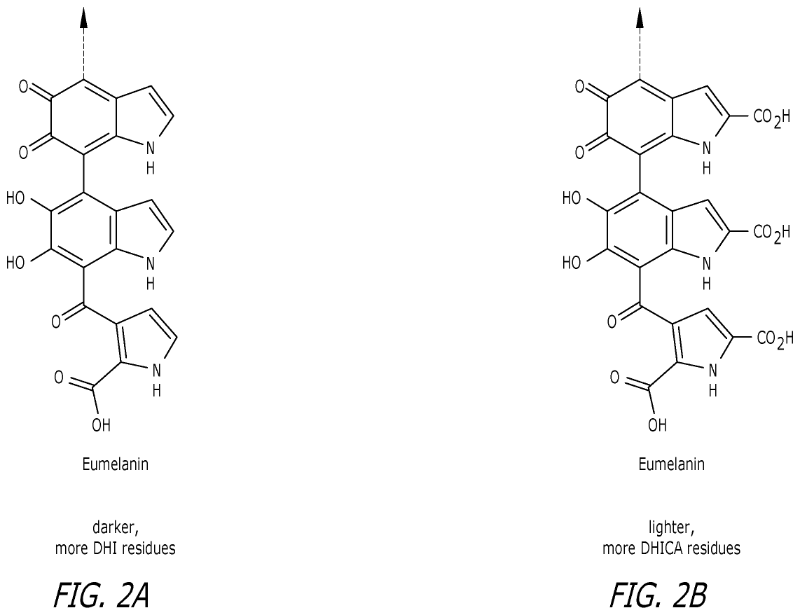

Taking, in view of the foregoing, levodopa as an example of the pigment precursor and both manganese gluconate and iron gluconate as examples of the transition metal catalysts, adjusting the ratio of manganese gluconate and iron gluconate in the pigment formulation in accordance with Table 1 can affect the oxidation of levodopa and, thus, distribution of DHI and DHICA in the pigment formulation. This, in turn, can affect polymerization of eumelanin as the distribution of DHI and DHICA controls the availability of DHI and DHICA for incorporation into eumelanin as residues thereof. Indeed, subsequent to oxidation of levodopa and polymerization of DHI and DHICA, the resulting eumelanin has residues selected from DHI and DHICA as shown in FIG. 1, the ratio of which residues can affect the level of hair color achieved by the hair treatment formulation. Continuing to embrace some theoretical flexibility, should eumelanin have more DHI residues than DHICA residues as shown in FIG. 2A, the hair treatment formulation including such eumelanin can achieve a darker level of hair color such as ≤2 on account of eumelanin having efficient π-electron delocalization and, thus, strong, broad absorption across the visible light spectrum. Table 1 shows that such a hair treatment formulation can result when the transition metal catalyst(s) include more manganese, particularly more than about 4:1 manganese to iron by weight of their gluconates. Should eumelanin have more DHICA residues than DHI residues as shown in FIG. 2B, the hair treatment formulation including such eumelanin can achieve a lighter level of hair color such as 4-7, inclusive, on account of eumelanin having less efficient π-electron delocalization and, thus, weaker absorption across the visible light spectrum due to the electron-withdrawing carboxylic acid moiety of the DHICA residues. Table 1 shows that such a hair treatment formulation can result when the transition metal catalyst(s) include more iron, particularly more than about 2:1 iron to manganese by weight of their gluconates.

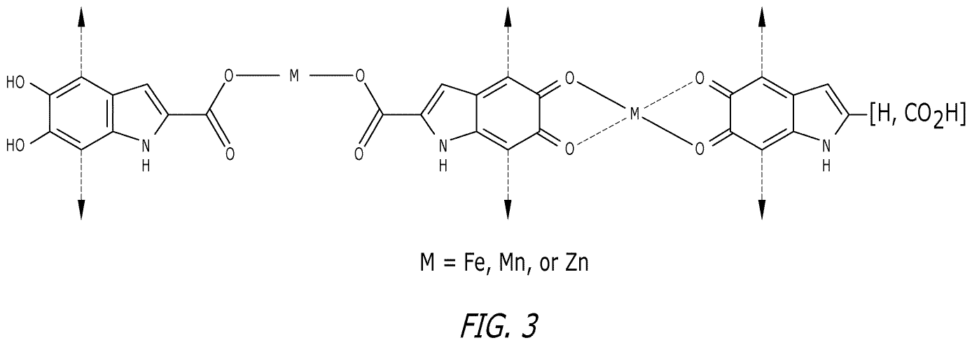

Continuing to embrace some theoretical flexibility, adjusting the species of the transition metal catalyst(s) in the pigment formulation, the ratio(s) of the transition metal catalyst(s) in the pigment formulation, or both can also affect a degree of oxidation of the residues in the pigment, wherein a higher degree of oxidation can, like that set forth above, result in more efficient π-electron delocalization and, thus, strong, broad absorption across the visible light spectrum. Further, adjusting the species or ratio(s) of the transition metal catalyst(s) in the pigment formulation can affect a degree of polymerization of the pigment, introduce variations in polymer structure of the pigment, cause supramolecular aggregation of the pigment, or a combination thereof, each of which can result in more or less efficient I-electron delocalization and, thus, better or weaker absorption across the visible light spectrum for a darker or lighter level of hair color with the hair treatment formulation. Lastly, as shown in FIG. 3, adjusting the species or ratio(s) of the transition metal catalyst(s) in the pigment formulation can affect interactions among the pigment and the transition metal catalyst(s). Indeed, FIG. 3 illustrates a couple of chelation modes in eumelanin as the pigment, which chelation modes can assist in the foregoing supramolecular aggregation of the pigment as well as promote electron delocalization in accordance with the species of the transition metal catalyst(s).

Further, it has been surprisingly found that kinetics of the oxidation of the pigment precursor to the pigment in the pigment formulation can be inhibited in accordance with the stabilizer(s) in the pigment formulation. With reference to the emulsion as the pigment formulation, this advantageously allows formation of the emulsion, application of the emulsion to hair, and appreciable oxidation of the pigment precursor to the pigment on hair shafts of the hair, as opposed to non-inhibited, diffusion-limited oxidation of the pigment precursor to the pigment upon formation of the emulsion, which also contributes to the level of hair color achieved by the hair treatment formulation. While embracing some theoretical flexibility, oxidation of the pigment precursor to the pigment on the hair bonds the pigment to keratin of the hair shafts of the hair, thereby contributing to the level of hair color achieved by the hair treatment formulation. Following on other factors postulated above that result in more or less efficient π-electron delocalization in eumelanin and, thus, better or weaker absorption across the visible light spectrum for a darker or lighter level of hair color with the hair treatment formulation, protein bonding between the keratin of the hair shafts and the pigment can also modify the absorption of visible light by the pigment. Indeed, oxidation of the pigment precursor to the pigment on the hair under basic conditions can lead to covalent bonding between the keratin of the hair shafts and the pigment through oxidative crosslinking, formation of Schiff bases (e.g., between a lysine residue of keratin and a quinone of eumelanin), formation of Michael addition adducts (e.g., between a cysteine residue of keratin and a quinone of eumelanin), or some combination thereof, which can extend or disrupt π-electron delocalization in the pigment. That, and the protein bonding between the keratin of the hair shafts and the pigment can also modify the absorption of visible light by the pigment by way of various non-covalent interactions including hydrogen bonding and other electrostatic interactions.

### Dispensing Devices

FIG. 5 illustrates a dispensing device 100 for the hair treatment formulation in accordance with some embodiments.

As shown, the dispensing device 100 is a dual- or double-compartmented dispensing device including a first compartment 102, a second compartment 104, and a mixing tip 106 to which each compartment of the first and second compartments 102 and 104 is fluidly connected in at least a ready-to-dispense state of the dispensing device 100. Notably, the mixing tip 106 need not be fluidly connected to each compartment of the first and second compartments 102 and 104 in a packaged state of dispensing device 100. Indeed, for optimal preservation of the hair treatment formulation during storage, each compartment of the first and second compartments 102 and 104 is sealed off from the other, for example, by way of independent seals or a common seal independently sealing each of the foregoing compartments. That said, the mixing tip 106 can still be coupled to a remainder of the dispensing device 100, as shown, thereby demonstrating in the packaged state of the dispensing device 100 a proper assembly of the dispensing device 100 in the ready-to-dispense state thereof.

Continuing with the pigment-precursor formulation as the oil-based dispersion and the oxidant formulation as the aqueous solution but with the understanding that the pigment-precursor formulation and the oxidant formulation are not limited to such mixtures, as set forth above, the dispensing device 100 can include the pigment-precursor formulation as the oil-based dispersion in the first compartment 102 of the dispensing device 100 and the oxidant formulation as the aqueous solution in the second compartment 104 of the dispensing device 100. Again, each compartment of the first and second compartments 102 and 104 is sealed off from the other for optimal preservation of the hair treatment formulation in the packaged state of the dispensing device 100. However, upon unsealing each of the foregoing compartments and fluidly connecting the mixing tip 106 thereto, thereby instantiating the ready-to-dispense state of the dispensing device 100, the dispensing device 100 is configured for combining the oil-based dispersion and the aqueous solution in at least the mixing tip 106 to form the emulsion including the pigment for hair treatment by way of a dispensing action corresponding to the dispensing device 100.

When the dispensing device 100 is a double-barreled syringe 108, as shown, the first compartment 102 of the dispensing device 100 corresponds to a first barrel chamber defined by a first barrel 110 of the syringe 108, the first barrel chamber including the pigment-precursor formulation as the oil-based dispersion. Likewise, the second compartment 104 of the dispensing device 100 corresponds to a second barrel chamber defined by a second barrel 112 of the syringe 108 conjoined with the first barrel 110, the second barrel chamber including the oxidant formulation as the aqueous solution. Such a double-barreled syringe 108 also includes a plunger 114 having a pair of plunger shafts 116 with a common plunger flange 118 but independently tipped with a pair of pistons (not shown) distributed therebetween. If necessary, the plunger 114 can be clipped or otherwise held in place relative to the first and second barrels 110 and 112 of the syringe 108 in the packaged state thereof. Upon instantiating the ready-to-dispense state of the dispensing device 100, as set forth above, the double-barreled syringe 108 is configured for combining the oil-based dispersion and the aqueous solution in at least the mixing tip 106 to form the emulsion by way of pushing the plunger 114 and, thus, both plunger shafts 116 simultaneously into the first and second barrels 110 and 112 of the double-barreled syringe 108.

Notwithstanding the foregoing, it should be understood the hair treatment formulation can be alternatively distributed between any combination of two single-compartmented dispensing devices selected from syringes, bags, tubes, or the like. Indeed, a first syringe, a first bag, or a first tube can include the pigment-precursor formulation as the oil-based dispersion, and a second syringe, a second bag, or a second tube, which need not be respective to the first syringe, the first bag, or the first tube, can include the oxidant formulation as the aqueous solution. Upon unsealing the two single-compartmented dispensing devices, the dispensing devices are configured for dispensing the oil-based dispersion and the aqueous solution in a non-reactive receptacle for mixing to form the emulsion including the pigment for hair treatment. However, in order to allow appreciable oxidation of the pigment precursor to the pigment on hair shafts of hair, the emulsion should be applied to the hair within any time period indicated on the packaging of the hair treatment kit or in the instructions provided therewith to achieve a predetermined level of hair color associated with the hair treatment kit.

### Hair Treatment Kits

A hair treatment kit includes packaging for components of the hair treatment kit, which packaging includes an indication thereon of a predetermined level of hair color that can be achieved with the hair treatment kit. The foregoing components of the hair treatment kit include at least one dispensing device 100 having the hair treatment formulation disposed in the dispensing device 100. In addition, the hair treatment kit can include one or more additional components selected from one or more packages of chelating shampoo for removing at least transition metals or other residues from hair prior to applying the pigment formulation, one or more tint brushes for applying the pigment formulation, gloves for preventing hands from becoming stained by the pigment formulation, and instructions for using the hair treatment kit if not provided on the packaging, the Internet, or the like. Notably, the instructions can include a time period within which the pigment formulation should be applied to hair if prepared in a non-reactive receptacle like that set forth above. Indeed, applying the pigment formulation to the hair within such a time period beneficially allows oxidation of the pigment precursor to the pigment on hair shafts of the hair for achieving the predetermined level of hair color associated with the hair treatment kit.

### Methods

Methods include methods of making and using any one or more components of the hair treatment kit up to an entirety of the hair treatment kit. In an example, the methods can include a method of making the hair treatment formulation as well as a method of making the hair treatment kit. In another example, the methods can include a method of using the hair treatment kit.

Continuing with the pigment-precursor formulation as the oil-based dispersion and the oxidant formulation as the aqueous solution, the method of making the hair treatment formulation can include making the oil-based dispersion and the aqueous solution, as set forth below.

When making the oil-based dispersion, the method can include mixing the stabilizer(s) into the oil(s), optionally, with heating, as appropriate. For example, the method can include mixing dextrin palmitate into coconut oil with heating to ensure the coconut oil remains a liquid for the mixing. The method can also include subsequently mixing any single component or combination of components selected from the pigment precursor, the transition metal catalyst(s), and the pH-modifying agent(s) into a nascent oil-based dispersion of the stabilizer(s) in the oil(s), thereby forming the oil-based dispersion of the hair treatment formulation. For example, the method can include subsequently mixing an admixture including levodopa, sodium bicarbonate, and manganese gluconate into the oil including the dextrin palmitate to form the oil-based dispersion of the hair treatment formulation. However, should the oil-based dispersion include the preservative(s) or the haircare ingredient(s) as set forth above, such components can be mixed into the oil(s), the oil(s) including the stabilizer(s), or that including the pigment precursor, the transition metal catalyst(s), or the pH-modifying agent(s), as the case might be.

When making the aqueous solution, the method can include mixing the oxidant into water. For example, the method can include mixing hydrogen peroxide (e.g., 3% aqueous hydrogen peroxide) into the water. The method can also include subsequently mixing the viscosity modifier(s) into the aqueous solution including the oxidant, thereby forming the aqueous solution of the hair treatment formulation. For example, the method can include subsequently mixing konjac gum into the aqueous solution including the hydrogen peroxide to form the aqueous solution of the hair treatment formulation. However, should the aqueous solution include the preservative(s) or the haircare ingredient(s) as set forth above, such components can be mixed into the water, the aqueous solution including the oxidant, or that including the viscosity modifier(s), as the case might be.

Following on the method of making the hair treatment formulation, the method of making the hair treatment kit can include preparing the dispensing device 100, wherein each of the oil-based dispersion and the aqueous solution of the hair treatment formulation is loaded into its corresponding compartment 102 or 104 of the dispensing device 100 and sealed therein. In addition, the method of making the hair treatment kit can include packaging the dispensing device 100 in the packaging along with the additional component(s), if any, selected from the package(s) of chelating shampoo, the tint brush(es), the gloves, and the instructions for using the hair treatment kit.

The method of using the hair treatment kit can include washing hair with a chelating shampoo to remove at least transition metals or other residues from the hair prior to applying any of the emulsion to the hair. When using the double-compartmented dispensing device 100, the method also includes dispensing the emulsion including the pigment for hair treatment from the mixing tip 106 of the dispensing device 100.

While the oil-based dispersion and the aqueous solution form the emulsion and at least some of the pigment in the mixing tip 106 during the dispensing from the dispensing device 100, appreciable oxidation of the pigment precursor to the pigment can occur after dispensing the emulsion from the dispensing device 100 depending on the stabilizer(s) set forth above.

Accordingly, the method can include dispensing the emulsion from the dispensing device 100 directly onto the hair and spreading the emulsion through the hair with the tint brush, thereby allowing appreciable oxidation of the pigment precursor to the pigment on hair shafts of the hair. However, some might find it beneficial to follow the method that includes dispensing the emulsion from the dispensing device 100 into a non-reactive receptacle to further mix it with, for example, the tint brush, thereby allowing oxidation of the pigment precursor to the pigment to substantially complete before applying the emulsion to the hair and spreading it therethrough with the tint brush. When using two single-compartmented dispensing devices instead of the dispensing device 100, the method likewise utilizes the foregoing non-reactive receptacle. Indeed, the method includes dispensing the oil-based dispersion and the aqueous solution in the non-reactive receptacle and mixing them, for example, with the tint brush, to form the emulsion and allow oxidation of the pigment precursor to the pigment to complete before applying the emulsion to the hair and spreading it therethrough with the tint brush. No matter the dispensing device or devices, the method also includes allowing the emulsion to age on the hair for a period of treatment time (e.g., 10 minutes) to effectuate the hair treatment with at least the pigment. Lastly, the method also includes rinsing the emulsion from the hair subsequent to the foregoing period of treatment time.

### EXAMPLES

### Example 1: Dark Brown Formulation

As the oil-based dispersion of the hair treatment formulation, 1.40 g dextrin palmitate is stirred into 33.52 g of coconut oil while heating between 8° and 130 °C. Upon cooling, an admixture of 17.28 g of M. pruriens, 1.8 g of manganese gluconate, and 15.84 g of sodium bicarbonate is stirred into the oil-based dispersion of dextrin palmitate and coconut oil to form the oil-based dispersion of the hair treatment formula.

As to the aqueous solution of the hair treatment formulation, 33.6 g of 3% hydrogen peroxide is added to 80 g of distilled water followed by 1.44 g of konjac gum. An overhead mixer at about 1200 RPM is used to mix the resulting aqueous solution for an hour to form the aqueous solution of the hair treatment formulation.

When the oil-based dispersion and the aqueous solution of the dark brown hair treatment formulation are combined to form an emulsion and applied to hair, as in the method set forth above, the color imparted or restored to the hair is described as Level 3: Dark brown in accordance with the hair color level system used across various professional hairdressing brands.

### Example 2: Light Brown Formulation

As the oil-based dispersion of the hair treatment formulation, 1.40 g dextrin palmitate is stirred into 33.52 g of coconut oil while heating between 8° and 130 °C. Upon cooling, an admixture of 18.58 g of M. pruriens, 0.43 g of ferrous gluconate, 3.21 g of sodium bicarbonate, and 12.70 g of sodium carbonate is stirred into the oil-based dispersion of dextrin palmitate and coconut oil to form the oil-based dispersion of the hair treatment formula.

As to the aqueous solution of the hair treatment formulation, 33.6 g of 3% hydrogen peroxide is added to 80 g of distilled water followed by 1.44 g of konjac gum. An overhead mixer at about 1200 RPM is used to mix the resulting aqueous solution for an hour to form the aqueous solution of the hair treatment formulation.

When the oil-based dispersion and the aqueous solution of the light brown hair treatment formulation are combined to form an emulsion and applied to hair, as in the method set forth above, the color imparted or restored to the hair is described as Level 5: Light brown in accordance with the hair color level system used across various professional hairdressing brands.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A hair treatment formulation, comprising:
a dispersion including:
a continuous phase including one or more oils; and
a dispersed phase including:
a pigment precursor;
one or more transition metal catalysts including at least manganese, iron, zinc, or a combination thereof, and
one or more stabilizers for keeping the dispersed phase dispersed in the continuous phase; and
an aqueous solution including an oxidant, wherein the dispersion and the aqueous solution form an oil-in-water emulsion for hair treatment upon combination with each other, the oxidant oxidizing the pigment precursor in accordance with species of the transition metal catalyst(s) in the emulsion, ratio(s) of the transition metal catalyst(s) in the emulsion, or both to provide a pigment for achieving a predetermined level of hair color with the hair treatment formulation.

2. The hair treatment formulation of claim 1, wherein oxidation of the pigment precursor to the pigment is inhibited in the emulsion by the stabilizer(s), thereby allowing formation of the emulsion, application of the emulsion to hair, and appreciable oxidation of the pigment precursor to the pigment on the hair as opposed to non-inhibited, diffusion-limited oxidation of the pigment precursor to the pigment upon formation of the emulsion.

3. The hair treatment formulation of claim 2, wherein oxidation of the pigment precursor to the pigment on the hair bonds the pigment to keratin present in hair shafts of the hair, thereby contributing to the predetermined level of hair color achieved by the hair treatment formulation.

4. The hair treatment formulation of claim 1, wherein the pigment precursor is a catecholamine and the pigment is a polymer having residues selected from various oxidation intermediates of the catecholamine.

5. The hair treatment formulation of claim 1, wherein the pigment precursor is levodopoa and the pigment is eumelanin having residues selected from those of 5,6-dihydroxyindole (“DHI”) and 5,6-dihydroxyindole-2-carboxylic acid (“DHICA”).

6. The hair treatment formulation of claim 5, wherein the pigment has more DHI residues than DHICA residues in accordance with the transition metal catalyst(s) including more manganese.

7. The hair treatment formulation of claim 6, wherein the predetermined level of hair color achieved by the hair treatment formulation is ≤2.

8. The hair treatment formulation of claim 6, wherein the transition metal catalyst(s) include approximately 4:1 manganese to iron by weight.

9. The hair treatment formulation of claim 5, wherein the pigment has more DHICA residues than DHI residues in accordance with the transition metal catalyst(s) including more iron.

10. The hair treatment formulation of claim 9, wherein the predetermined level of hair color achieved by the hair treatment formulation is 4-7.

11. The hair treatment formulation of claim 9, wherein the transition metal catalyst(s) include approximately 2:1 iron to manganese by weight.

12. The hair treatment formulation of claim 1, wherein the oil(s) are selected from coconut oil, fractionated coconut oil, olive oil, argan oil, avocado oil, jojoba oil, sweet almond oil, grapeseed oil, baobab oil, castor oil, macadamia nut oil, marula oil, and sea buckthorn oil.

13. The hair treatment formulation of claim 12, wherein the oil(s) include coconut oil or fractionated coconut oil.

14. The hair treatment formulation of claim 1, wherein the pigment precursor is present in the dispersed phase as a constituent of Mucuna pruriens seed powder or an extract thereof.

15. The hair treatment formulation of claim 1, wherein the stabilizer(s) are selected from dextrin palmitate, sucrose laurate, and dibutyl lauroyl glutamide.

16. The hair treatment formulation of claim 15, wherein the stabilizer(s) include dextrin palmitate.

17. The hair treatment formulation of claim 1, wherein the oxidant is hydrogen peroxide.

18. The hair treatment formulation of claim 1, wherein the aqueous solution further includes one or more viscosity modifiers in a concentration suitable for modifying a viscosity of the aqueous solution.

19. The hair treatment formulation of claim 18, wherein the viscosity modifier(s) include konjac gum.

20. A hair treatment kit, comprising:
a dispensing device including a first compartment and a second compartment;
a dispersion in the first compartment, the dispersion including:
a continuous phase including coconut oil or fractionated coconut oil; and
a dispersed phase including:
a constituent of Mucuna pruriens seed powder or an extract thereof as a pigment precursor;
manganese, iron, zinc, or a combination thereof as one or more transition metal catalysts; and
dextrin palmitate as a stabilizer for keeping the dispersed phase dispersed in the continuous phase; and
an aqueous solution in the second compartment, the aqueous solution including:
an oxidant; and
konjac gum in a concentration suitable for modifying a viscosity of the aqueous solution, wherein the dispersion and the aqueous solution form an oil-in-water emulsion for hair treatment upon dispensation from the dispensing device and combination with each other, the oxidant oxidizing the pigment precursor in accordance with species of the transition metal catalyst(s) in the emulsion, ratio(s) of the transition metal catalyst(s) in the emulsion, or both to provide a pigment for achieving a predetermined level of hair color as indicated on packaging of the hair treatment kit.
